(19) United States
(12) Patent Application Publication
(10) Pub. No.: US 2023/0410223 A1
(43) Pub. Date: Dec. 21, 2023

(54) DIGITAL PLATFORM FOR HEALTH USERS

(71) Applicant: RYKOV LLC, Dacula, GA (US)

(72) Inventors: Owen N. Dobson, Dacula, GA (US); Melissa K. Dobson, Dacula, GA (US); Austin S. Dobson, Dacula, GA (US)

(21) Appl. No.: 18/241,284

(22) Filed: Sep. 1, 2023

Related U.S. Application Data
(63) Continuation-in-part of application No. 16/634,896, filed on Jan. 29, 2020.
Application No. 16/634,896 is a continuation of application No. 16/392,623, filed on Apr. 24, 2019.
(60) Provisional application No. 62/661,163, filed on Apr. 23, 2018.

(51) Int. Cl. G06Q 50/00; G16H 15/00; G16H 10/60; G16H 80/00

(52) U.S. Cl. CPC G06Q 50/01; G16H 15/00; G16H 10/60; G16H 80/00

(57) ABSTRACT

The system disclosed allows a user to specify an illness, disease, or health condition, mental or physical or otherwise, and interconnect with other users having similar diagnoses, symptoms, conditions, complications, treatments, or otherwise. The patient user may be connected to a caregiver who links into the system to connect with other caregivers as well. The digital platform is a health web designed to interconnect patients, friends, families, doctors or others who seek personalized medical information pertaining to the experiences of others, creating models for treatment of persons within and external to medical settings. Such models may be real or simulated by artificial intelligence (AI). In creating decision trees within the digital platform, real and synthetic data can be incorporated. The system creates an AI-driven large learning model (LLM) to impact persons health and provider care, globally.

### CROSS-REFERENCES TO RELATED APPLICATIONS

The present application is a continuation-in-part application of U.S. patent application Ser. No. 16/634,896, filed Jan. 29, 2020, which is a U.S. national stage patent application (a “371 application”) of a PCT patent application no. PCT/US19/38540, filed Jun. 21, 2019, which claims the benefit of priority (and with right to restore priority accepted) from U.S. provisional patent application No. 62/661,163, filed Apr. 23, 2018. Further, the PCT patent application no. PCT/US19/38540 also claims the benefit of priority from U.S. patent application Ser. No. 16/392,623, filed Apr. 24, 2019. The entire contents of each of the foregoing are each hereby expressly incorporated by reference into this disclosure as if set forth in its entirety herein.

### FIELD

The present invention relates generally to interconnecting the health data of patients and data respective of professional experiences of medical providers, globally as to both, and more particularly, to a system and method for extrapolating data from real-time data collections systems to analyze the data points, and to create machine learning and artificial intelligence driven digital decision-support to a mobile device for both patient and medical provider.

### BACKGROUND

Current social networks allow users to interface based on common daily interactions such as exchanging photos of food, to the latest hairstyle, children's videos, and social event planning Such social networks such as Twitter, Facebook, Tumblr, Instagram, WhatsApp, SnapChat, among others, fail, however, to have a professional interface that extends beyond common social interactions. Even dating apps such as Match, Bumble, eHarmony, and numerous others available fail to interconnect persons beyond identified personal interests and desires.

Social network services typically consist of online communities of individuals or groups of individuals who share a common background, attributes, interests and/or activities, and who are interested in meeting and/or interacting with other individuals in the network. Most social network services are web-based and provide a variety of ways for users to interact, such as via email, instant messaging, posting blogs, and posting comments on each other's social network profile pages. A number of social network services have developed solutions to accommodate users participating in social networks through the use of wireless devices, and other portable electronic devices.

Whether for dating, friendship, activities, deal-making, or reuniting, conventional social networking solutions, such as online social networks, typically require a user who wants to find other members that share similar interests to designate the specific attributes sought at the time the user wants to find these members. It is often difficult to find users with desired qualities because conventional social networking solutions typically have many users, and entering desired attributes often returns too many potential matches. Searching for other members that a user would find of interest oftentimes requires sorting through the profiles and data of many other members and/or performing multiple searches to find individuals of interest. Users of many conventional social networks may also search for individuals that one may have interest in by scanning though the profiles and data of users associated with already-known members. In some instances, meeting individuals who have an established relationship with an already-known individual may require a user to request permission from a users already-known contact, the person of interest, or both. This results in a delay for the user before the user can meet the person of interest as well as additional user effort. Further, although a user may find another member in a social network desirable and may want to interact with that individual, it is often difficult to determine if the user himself or herself has attributes that the other individual is seeking.

Meanwhile, currently existing health programs, legalities of government, and community systems interfere with connecting resources for individual patients, his/her family and friends and the associated medical providers. Even internal hospital and medical systems, and the software utilized by medical providers, fail to interconnect medical professionals across the globe, or even within a community or the same city. Patient information remains situated within encrypted servers to protect patient data, and respectfully protect privacy via regulatory controls. The systems lack a consistent language or synchrony of software across medical servers, restrict access, and/or do not provide sharing capabilities among patients. Patient portals or any internal medical information remains locked in private accounts.

In addition, medical providers connect through medical associations, annual continuing medical education activities, fundraising, among other in-person servicing, but rely solely on word of mouth and attendance at events to expand knowledge capabilities and interconnections. Even online resources are just that—online, without personalized access to patients or medical providers outside their individualized education or professional associations.

The unmet needs of current social networking are not on target with the needs of patients and professionals in medicine and healthcare. While a user may seek out a community that has a targeted illness to connect personally, the community lacks an infrastructure to aid, assist and facilitate diagnoses, resolving symptoms, interconnecting communications as to treatments, medicines, access to resources, funding, or expertise in medicine. Further, while patients are not able to find resources via social networks, professionals in medicine, such as the medical provider, doctor, surgeon, nurse practitioner or other health professional is not able to interconnect with medical professionals globally, or in confidence, unless a prior connection is known from prior encounter or via word of mouth.

A need exists for an electronic system, a platform that can personalize decision support to a patient, to families and friends of loved ones who desire information upon patient diagnosis to properly resource their needs for knowledge of not only the disease, but including personalized connections to others who suffer and heal similarly. The platform will beneficially be able to share data sets publicly across the network to create patient authorized interconnection with others, or medical professionals globally, and/or obtain comparisons in medicine from other patients and/or medical professionals. In turn, the participation of medical providers or health professionals in the platform, will create data personalized to care providers who have particular education and licensure, as well as experiential learnings from their own professional practice of medicine or the specialized health field, so as to create databases that will analyze the data from the professional sector and integrate with patient data to create a personalized treatment recommendation. As well, the medical professional network will allow a doctor, surgeon, nurse or otherwise to expand particular knowledge base(s) as to diagnosis and treatments across the world's medical communities by taking into account the differences in learnings, education, background, and professional care, or in clinical medicine. The following will beneficially detail the possibilities of solving such needs and implementing the technological measures to achieve the same.

### SUMMARY

High speed networks across the web and cloud computing have evolved into interconnections to facilitate networks around the globe. The health network system described herein resolves the issues described above by addressing the professional use of social networks and real-time access to patients and medical professionals around the world.

The network of healthcare resources (1) targets market needs (customers), (2) utilizes federal and state funding initiatives (economic growth), and (3) revolutionizes medicine through innovation, translational research (clinical focus), commercializing and updating data systems for predictive analytics and deep learning for easy access data in healthcare delivery.

The health networking system and methodology targets the personalized and global community needs in medicine and healthcare to interconnect information as to diagnosis, symptoms, treatments, side-effects, and chronic and acute conditions, among others, further addressing short-term and long-term effects and recurrences. The network seeks out individual users in a community or across the globe with similar ailments or medical conditions, and may include those users with professional expertise. While a user may seek out another individual or community that has a targeted illness to connect personally, the community is formed via a computer-based infrastructure that facilitates the network connections of users. The users can correspond as to diagnosis, diseases, symptoms, treatments, side-effects, use of particular medicines and/or natural supplements. The system provides access to resources, funding, and expertise in medicine as well.

The health network system allows patients to find resources via social networks, professionals in medicine, such as the medical provider, doctor, surgeon, nurse practitioner or other health professional, and suggests possible interconnects across the globe, either with other patients, family members, and/or medical professionals. The communications are private as selected via a user profile and shared with those who desire to interconnect. Any privacy information shared is therefore at the discretion of a user and not subject to regulatory laws where a patient provides details of his/her condition.

The electronic system is a health social network that can interconnect patients, families and friends of loved ones who desire information upon diagnosis to properly resource their needs for knowledge, specifically personalized knowledge from not only online resource dictionaries, but directly from patients or those being treated for similar conditions, from those who are also suffering from an ailment, or healing from disease. This emotional support network satisfies deficiencies in current social networks. In addition, social networks have failed to accommodate the mental health communities, such as those with post-traumatic stress syndrome (PTSD) and stages of depression; the current health social network provides for a network to confidentially communicate and manage emotional, psychological, and psychiatric needs to better mental health for patients and quality of life. Furthermore, users with mental health conditions can discover individuals with similar ailments, concerns, or even discover friendships to alleviate the unsatisfactory conditions that cause deficient or unhealthy mental states.

Further, the health network is a progressive online network to focus on individual and globalized health needs, studies in epidemiology, medical education, and professional development. Data collection and analytical systems will utilize the data to predict outcomes, treatments, disease, utilizing predictive analytics, machine learning, deep learning, and detailed to expansive artificial intelligence (AI) social networking health system.

The system utilizes the connection with medical professionals including doctors, surgeons, nurses, nurse practitioners, pharmacists, podiatrists, psychiatrists, among others to connect with his/her specific patient and interconnect on a backend of the network with the doctors or medical professionals interconnected with patients who have opted into the network. The health social network therefore allows medical professionals to post, message, or provide advice, if practicable, and to post, message and directly connect with another medical professional of the interconnected patients to build an expanding collegial network.

The individuals of the social network of patients will beneficially share their experiences publicly across the network to interconnect with others, allowing medical professionals to interconnect globally within ethical constraints. The system not only expands upon a network with personalized and comparison medicine; the system factors in attributes such as geography and environmental characteristics which could impact epidemiological studies and address global health conditions. Additionally, the remote and mobile access allows interconnection across the globe, within urban high tech settings to remote locations of the world with limited access to medical care and high end and/or affordable treatments. The health social network is a life line communication network to reach out to others with similar health conditions, from new moms to guardians caring for elderly parent, chronic conditions to acute disease, common illness to end-of-life care.

Operationally, the online health network can manage internal hospital social interactions among patients, physicians, hospital personnel, and further manage and review workflow. The data collected from the operational systems, including insurance management and payment systems, electronic medical records, and any of the above data entered by users to the system (i.e. patients, visitors, personnel, professionals, administrators, etc.) is then utilized in predictive analytics and AI to efficiently and effectively control costs in the delivery and management of healthcare, along with delivery of improved patient care through consistent decision-making and hospital authorizations.

Finally, the system shares and exchanges knowledge of Western and Eastern approaches of medicine, from the natural practices of the Amazon to the rainforests of Papua New Guinea, the reservations of Native Americans, North and South American medical practices, the practices of traditional chinese medicine (TCM), implementations of Ayurveda of India, and others known and unknown. From practices of medicine more than 3,000 years ago, utilizing concepts of health and disease that promote the use of herbal compounds, special diets, and other unique health practices, Eastern medicine can truly be implemented with Western practices.

By providing a connection across the hemispheres to remote and well-established regions of the world, patients and medical providers can now truly interconnect. Connections not known prior will be formed, allowing a revolutionized practice of medicine beyond the influence of politics and political regimes, beyond the influence of monetary wealth, and innovate all walks of life.

One embodiment is disclosed as a digital platform comprising one or more servers including a plurality of health data; one or more processors programmed to create profiles of one or more users, each profile including data tailored to a designated person, wherein the data includes parameters input by the user and extracted data from digital profiling, wherein the parameters comprise user demographics and health status; and a shared database directed by the server to store the data and the updated data; wherein the server is programmed to: (a) receive a request from the user at a user interface to create or join at least one online patient community categorized by one or more health conditions, (b) receive a request from the user to delineate personal identifiable information (PII) and de-identify the PII as de-identified data, (c) receive a request from the user to authorize release of the de-identified data to the shared database, and (d) access the data by way of the processor, wherein the processor uses machine learnings and artificial intelligence (AI) driven analytics to predict outcomes, treatments, and disease progression using user-derived data. The health status comprises one or more symptoms, complaint, injury, health condition, health status, disease-state, treatment, surgery, therapy, or medication of the user. The extracted data comprise social interactions, digital engagement across cloud-based platforms, metadata, cookies, reactions to digital media content, time spent with online communities, data entered by the user from the electronic medical record (EMR), and data from medical devices, wearable devices or implanted devices, the processor further programmed to update the data. In one aspect, communication between users occurs at the user interface external to a health provider portal. This is in reference to current health provider portals subject to U.S. regulations under HIPAA which applies to third party handling of patient data. Since the data here is in the control of the patient, the patient must authorize and release such handling of data manually or digitally.

In one aspect, the user of the digital platform can select or deselect the extracted data which will be integrated in the analytics of the processor. The plurality of health data comprises data input by a plurality of users and data aggregated from external resources, clinical studies, and clinical practice. Such data may come through external databases of the NIH, clinical research studies, epidemiology studies, among others.

Embodiments of the digital platform may set up synthetic data extraction. For exemplary purposes, one profile includes synthetic data artificially generated from real patient data to create an AI profile. In such case, the AI profile represents an artificially represented person (ARP) or may mimic a real person, or doppelganger, per se., i.e., augmented reality or virtual avatar. The parameters entered by a user may also comprise an election of one or more licensed health providers associated with the user. In such a case, the licensed health provider may be any care provider including individuals or system entities, including medical doctors, doctors of osteopathic medicine, dentists, specialists, surgeons, nurses, physician assistants, dental assistants, alternative care providers, aestheticians, hospital systems, health-affiliated educational institutes and organizations, managed care living facilities, mental health organizations, and other care provider groups. In fact, the licensed health provider need not be ‘licensed’ but may be accredited or authorized, such categorization and classification determined by geography and/or jurisdiction.

Embodiments disclosed herein allow a user to digitally request an electronic medical record (EMR) from the licensed health provider, the user authenticates the request, and the licensed health provider directs an uploaded version of the EMR to the user interface of the digital platform. In one aspect, the licensed medical provider may be provided a digital request to engage with the digital platform at a secondary user interface, and a secondary processor accesses the shared database to drive predictive analytics at a secondary server exclusive to a plurality of licensed health providers. In another aspect, the licensed health provider map provide inputs at the secondary user interface such as provider demographics, education, residency, clinical experience, and authorizes release of de-identified medical data from persons or machines to the shared database.

Embodiments herein include methods of using the digital platform. The system may be stand alone or may be integrated as a supplemental system to a health provider network. As well, the system may be an application that supports direct access of a user to his/her health data to others with similar health conditions, symptoms, ailments, and/or those seeking similar therapy or treatment. In one embodiment, the method encompasses using the digital platform by: initiating the user profile; defining a relationship between a plurality of users and the health status; creating one or more communities based on the demographics of the users, the user inputs, the health status, and associated with one or more particular health providers; and selecting search terms to generate artificial intelligence (AI) driven response to guide decision-making. Preferably, the data from the profiles is de-identified to synthesize simulated health conditions. The processor is then capable of generating a plurality of synthetic data from profiles with low risk and in protection of personally identifiable information (PII). In one aspect, the synthetic data creates at least one AI profile that represents an artificially represented person (ARP). As such, embodiments of the digital platform can simulate health conditions to create hypothetical clinical experiences to engage with a trainee, such as a medical student. The trainee can elect one or more treatments to structure a care plan for the user with a specified health condition. The training simulation creates an environment of low risk to health settings, particularly to patients, but also conserving resources and lowering costs (especially given the excessively escalating costs of drugs, use of medical devices, imaging, surgeries and other treatments/therapies). Embodiments of the simulation further comprise simulating health conditions structured in an artificial intelligence (AI) created profile that respond to one or more AI generated treatment plans, and generate graphical depictions representing risk and success rates corresponding to the AI generated treatment plans. The illustrations create decisive action and reduce time by creating a depiction that requires a decision be made at a targeted time and within a specified task of the treatment plan.

The details of the planned treatment and simulation may be modified by algorithms or enhanced imaging capabilities, as known in the art. Such improved technological capabilities, e.g. color contrasting, more refined graphical depictions or improved mathematical models, are encompassed in the heart of the disclosure herein.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. The various features are not necessarily drawn to scale. In fact, the dimensions may be arbitrarily increased or decreased for clarity of discussion. In the drawings:

FIG. 1 is an exemplary schematic as to an overall health network.

FIG. 2A is a schematic representing a patient interactive social network.

FIG. 2B is a depiction of an embodiment of a patient user interface.

FIG. 3 is a schematic as to a backend interconnection of medical providers in a health network described herein.

FIG. 4 is a graphical depiction of an embodiment that captures a presentation of selected data from the database and implements machine learning algorithms and predictive analytics.

FIG. 5 illustrates an exemplary flowchart as to engaging interconnections within a health network.

FIG. 6 methodology depicts operations of the network to include collection and integration of client [patient] user.

FIG. 7 assimilates information from a medical query to further engage a medical provider as a client user.

FIG. 8 represents system development of databases, libraries of information, and creation of communities.

FIG. 9A corresponds to value add-ons in the system, such as recommendations, advertisements, availability of clinical trials, patients receiving particular pharmaceutical drugs, among others.

FIG. 9B represents a patient user interface in one embodiment of the system.

FIG. 10 illustrates an embodiment of the social health network whereby a family member and/or friend interconnects with the patient client-user, or can set up an ‘echo’ or shadow profile for a known patient that can request patient join.

FIG. 11 illustrates an embodiment of a doctor profile.

FIG. 12 depicts an embodiment of various users in a defined community within the social health network.

FIG. 13 illustrates a representative social health system of communities, possible to be configured selectively and/or automatically.

FIG. 14 is a graphical depiction of disease regionally, and spatially in time, targeted to a specific disease.

FIG. 15 represents an embodiment in a geographical depiction representing aspects of the information defined herein.

FIG. 16 represents an embodiment in a depiction of efficacy of treatment aligned with demographics in one aspect.

FIG. 17 illustrates an embodiment encompassing synthetic data.

FIG. 18 illustrates an embodiment that creates real and simulated treatment plans.

### DETAILED DESCRIPTION

Reference will now be made in detail to the present embodiments of the invention, examples of which are illustrated in the accompanying drawings. Wherever possible, the same reference numbers will be used throughout the drawings to refer to the same or like parts.

The health social network provided herein encompasses an individual patient, his/her family, friends, and/or associated medical professionals. While implemented regionally to obtain comparisons in medicine from other patients and/or medical professionals, the system is utilized globally to encompass and assimilate a volume of data not currently available in the health market without individual integration of medical systems. The data is patient managed and authorized for release to medical professionals (e.g. medical, dental, nursing, affiliated health professions in physical therapy, respiratory therapy, occupational therapy, or otherwise). As well, the medical professional network will allow a doctor, surgeon, nurse or otherwise to expand his/her individual knowledge base as to diagnosis and treatments across the world medical communities; and/or allow a health system to consistently manage symptoms as aligned with diagnoses, treatment options, alternative care and care plans. Algorithmic analyses integrates, collects, and analyzes the data based on the unique qualifiers, inquiries, and coding selected in the backend user and medical provider systems, both component parts integrated as one and accessible individually by cleared and qualified users. The following will beneficially detail the possibilities of solving such needs and implementing the technological measures to achieve the same.

A system disclosed herein is a health targeted social network that utilizes social interactions and connections of patient users to better understand disease, illness, symptoms, treatments, and side effects. The system pulls together a patient's associated medical providers, allowing providers to connect on a back end confidential portal, implement other resources such as clinical trial availability, global treatments and protocols, among others. Various interactions and connections are possible, the perspective view allowing patients and loved ones (i.e., friends and family) to seek out personal connections around the world with those having particular ailments, disease, or perhaps deciding on treatment options as based on another patient's particular reactions and experiences. The following is not limited to the described interactions and may be broadened to expand within privacy (HIPPA) approved domains, pulling in external databases, implementing user interfaces, changing the patient or client user to any friend, family member, researcher, clinician, medical provider, service or insurance provider, among others.

Of particular mention and novelty of the online health social network is the placement of healthcare in the patient's control; access to a patient's data is authorized by the patient or the family member or agent with authorization to release patient data. Such relationship that is frequented here is parent—child where the child illness would prescribe a parent login to the system to validate child medical data for medical provider access (at provider's option/participation) in a confidential portal for medical providers, and an emotional and social network for a parent to discuss symptoms, diagnosis, treatment plans, drugs prescribed, interactions with other drugs, behaviors, and other details related to a child's health. The parent's interaction with other parents globally, delivers a personalized educational background to personalize a patient's treatment plan, expand options, offer alternatives from one end of the globe to the other, from Western medicine to Eastern medicine illnesses to treatments and curative programming

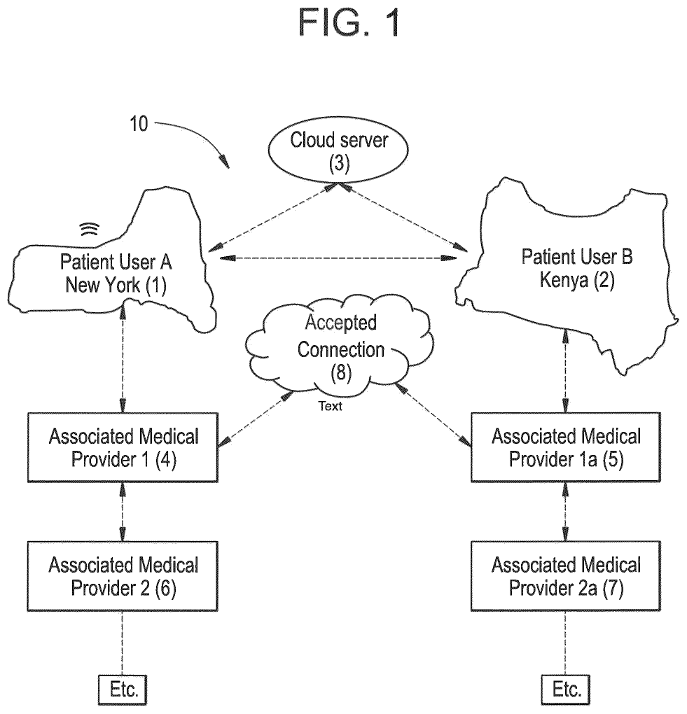

FIG. 1 illustrates an overview of an online social health network 10 facilitating network interaction between patients in different geographic locations. A Patient User A in New York State (1) creates a login and links to a Patient User B with similar medical condition (e.g. lung cancer) in Kenya (2). The cloud server 3 confidentially collects and stores the patient data. Patient User A provides information as to an associated Medical Provider 1 (4) while Patient User B provides his/her data to medical providers, here including an associated Medical Provider la (5). A number of medical providers can be associated including Medical Provider 2 (6), Medical Provider 2a (7), etc. The medical providers associated with each patient are then sent requests (as based on information provided by the associated patient and/or verified in a database that pools all databases of licensed medical providers including physicians, doctors, nurses, podiatrists, psychologists, clinicians, among others). A first licensed medical provider of a first associated patient will be authorized to connect with a second medical provider of a second associated patient, given the patient's authorization and consent via a disclosure process when setting up individual patient client-user profiles.

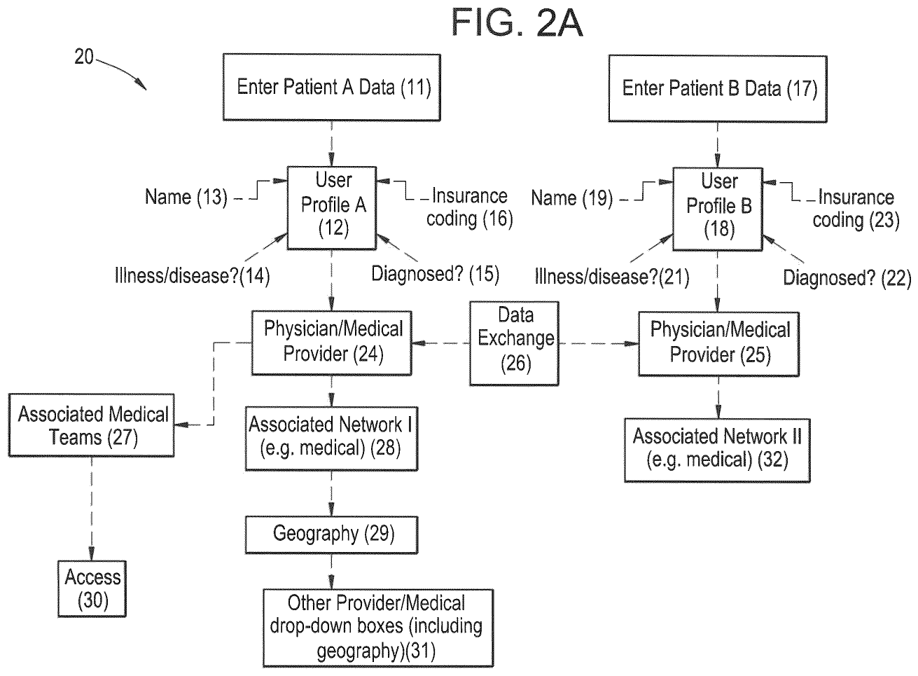

An online social health network system 20 is presented at FIG. 2A. Patient A enters data at data platform 11 to set up his/her patient profile 12 including name 13 and/or alias, background and/or medical history as voluntarily provided. Illness/disease 14, diagnosis 15, treatment and/or treatment options are also entered, and any data as so designated or requested in coding the platform for data entry. Patent B enters data at data platform 17 in a similar manner to establish a patient profile 18 as well, including name 19, alias, and detailed demographic information. In order for the health social network to operate, patients will voluntarily provide at least one or more symptoms, category of illness or disease 21, whether or not a diagnosed disease 22, treatment or medicine usage, among other data. This provides the interconnected tangible medical and health component to link client users and offer emotional support, educational knowledge from other patient's personal experiences, allow medical providers to discuss a patient's condition, behaviors, etc. The physician-to-physician contact or medical provider to medical provider communications occur on a shadow account (‘Shadow Chart’), in other words, a backend user interface (UI/UIX) that reflects patient information, but limits view specifically to medical providers 24, 25. As such, associated medical teams 27 are formed, allowing a ‘community’ for medical providers to share patient data at data exchange 26 to include charts, histories (again, with privacy protections and full disclosure to advise patient user as to sharing of any information provided, included release of medical histories to medical providers in order to co-develop solutions globally). Without having a better understanding of patient epidemiology, particular circumstances and situations globally, in a more personalized and centralized universe of data, medical solutions will remain isolated by geographies and resolutions left undiscovered. The universal application of the social network here provides not only social interactions and support in illness and disease, but also medical expertise on a unified global network that protects patient's records while seeking alternative and various viable treatment options. The wealth of information shared and explored further allows a medical provider to assist in treatment of his/her patient while traveling or to assist with another provider's patient on the other side of the world (or even in a neighboring state or health system).

An associated network 28 is a medical network such as EPIC within one hospital system, indicating a geographic region 29. The medical provider networks (e.g. 24, 28, 31) interconnect via an access point 30 which may be stored on a server or cloud based, with confidential and secure cyber measures. Other medical provider networks 31 may also tie in as populated in a database and selected in drop-down menus (including geographic region, locality, etc.).

A patient user interface (UI) 31 is illustrated at FIG. 2B to include user profile information 32, illness/disease 33, name/alias/location 34, diagnosis 35, and course of treatment 36, treatment plan/alternatives 37, medical provider listing 38 or association network. The patient UI 32 also includes a messaging capability 39 in real-time as well as suggested connections, daily updates/postings 40, events, descriptions, details and suggestions as to recommended ‘communities’ within the network, ads seeking patients for clinical trials, medicines currently utilized in treatment, among others. A translation tab 41 also is available to ensure communication across global communities.

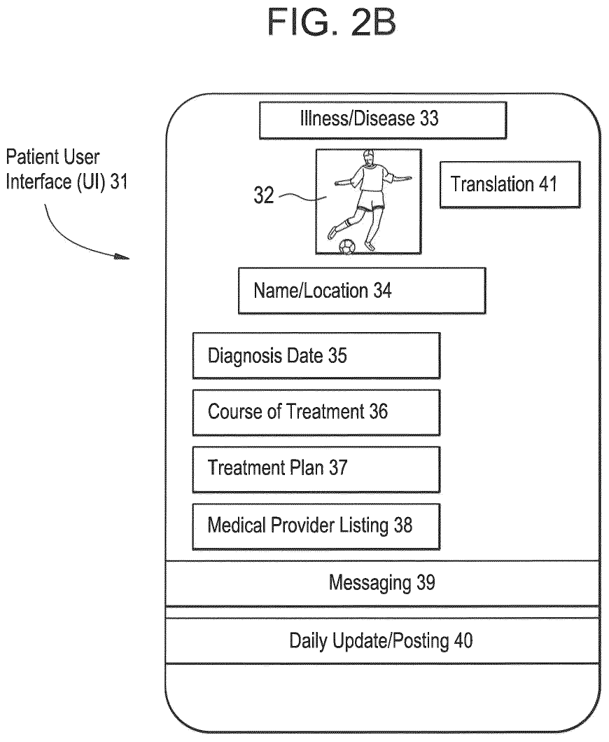

FIG. 3 depicts a medical provider user interface (UI/UIX) 50, here, that of a physician based in the United States, and licensed in the U.S. The physician client-user UI 50 provides the name 51 and title 52 of the medical provider (e.g. General Surgeon, Oncologist, etc.), location 53, medical service/network 54 or health network he/she associates or is employed by, image/picture 55, geographic map 56 of locations served (i.e., patient locations mapped having similar illness/disease and doctor location), medical specialty information 57, diagnosis typical 58 (e.g. as designated by symptoms in database and generated by AI and data analytics), treatment options offered 59, and patients 60 associated therewith (e.g. names using aliases set-up and configured by the patient during patient's entering of information during initial configuration). Note that aliases, or de-identification numbers may be associated with a patient, as preferred and designated by the patient in initial profile set-up and disclaimer regarding data usage and analytics. The patient information 60 and geography 61 are selections for data analytics to pool and configure selections of data across the globe as to specified and personalized UI/UIX for physician guidance and consistency of care, as well as expanding knowledge base for diagnosis and broadening treatment options. Treatment options as associated with ‘cure’ rates and defining ‘cure’ may also be designated by physician and/or health system to determine personalized treatments and care for patients and goals for patient, provider, health system, insurance provider, operations, and government entities.

The doctor/physical UI 50 is similar to any of medical provider UIs that are viewable to the private physician client-users as access points to the network 10. The physician here can then associate, on a backend database, aliases with patient medical records, EMRs if authorized by a medical center having discretion in sharing HIPAA outside the medical division for medical provider use only. The physician can then confirm participation in the network to at least access patient profiles, see patient information pulled via patient portals and modify/comment as to any inaccuracies, or perhaps suggest/post as appropriate. Note that the allowance of medical provider access keeps medical providers in contact to share in patient information, data, and experiences with particular patients, etc.

While the social network operates to connect medical providers on a backend to share and grow knowledge of disease and treatment options, medical providers may also seek assistance from other medical providers as to diagnosis of a patient for any rare disorders (e.g., ‘zebras’). The system may also gauge the involvement of a newly educated medical provider or physician who has limited clinical experience, and typically relies on book-based studies. The social health network system can personalize medicine and provide a larger scale community based medical program that takes into consideration vaccinations, epidemiology, environmental condition/circumstances, cultural practices, and details that may not be integrated with a patient's medical chart. Further, patients are much more willing to share experiences with one another than with physicians and/or nurse practitioners who are strangers to their personal lives.

As shown in FIG. 3, patient entered data 62 appears on one screen. The patient portal 63 allows the patient to authorize release of medical and confidential health information, electronic medical/health records (EMRs/EHRs) and scanned written records, via privacy disclaimers and compliant with HIPAA and regulatory measures. The physician and/or medical provider may modify the data of the patient as entered or uploaded via the authorization by patient to ensure accuracy or validity, perhaps correction or clarification which can be conveyed to the patient as well. The medical provider may interact with the patient in private messaging portal as desired.

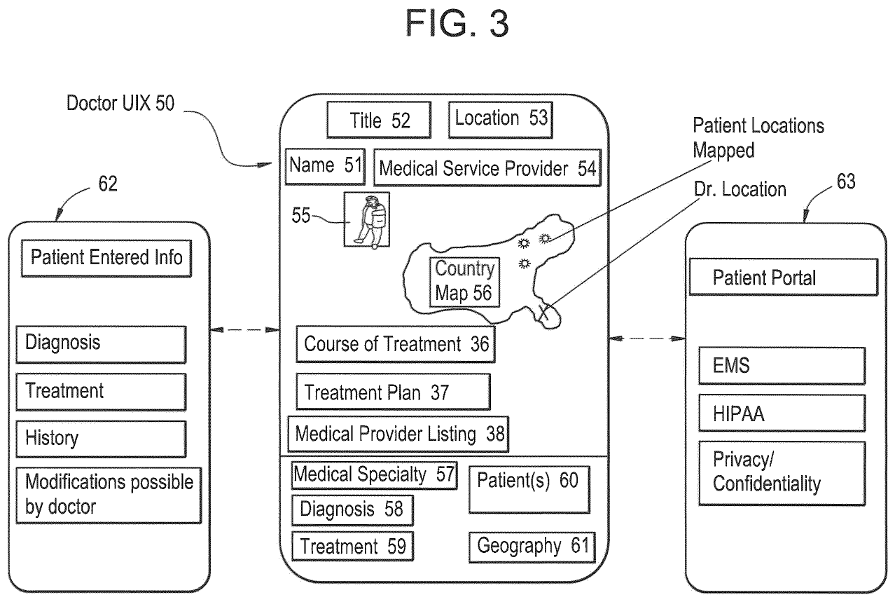

In FIG. 4, a medical provider has access to one or more databases 70 that pools patients by alias created to look at data across time, geography, injury, recovery periods, treatments, symptoms, age, demographics, etc. Prognosis and diagnosis may also be integrated to include machine learning once data has accumulated, and predictive analytics capabilities develop to further grow the functionality and utility of the network to experienced medical providers, as well as to patients seeking social engagement and support networks in critical care decision-making

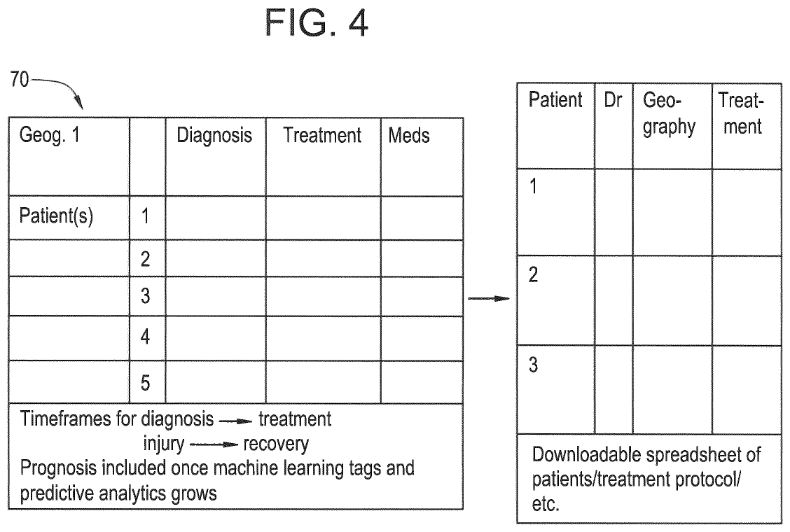

FIG. 5 is an embodiment of the system disclosed herein describing set-up and usability of a patient interface 501 of a social health networking application 500. The website is available via mobile app or accessible remote from a desktop. The patient interface 501 includes an application (app) 502 that is downloaded to a remote device wherein a user profile 504 is created by entering a username, user information, creating an alias or nickname for back-accessed records by medical providers linked in the system. The patient user links friends and/or family members 506, colleagues, persons, and entities that he/she desires to share medical/health information, status, and updates with. The patient user then names health conditions 508, including medical history 510, ailments including, for example, symptoms 512, disease/injury 516, diagnosis 518, followed by in query to treatments and treatment protocols 520, among others. Drop down or menus provide options from a dictionary, the data entered creating, comparing, and selectively storing data in the dictionary as determined by algorithmic variations of terms, existing libraries of information, etc. If a disease or injury is selected, the flow chart proceeds to a next step of inquiring as to diagnosis, medical history, symptoms, and other variable informative details, inclusive of behaviors, activity levels, drug use, social interactions in society, among others. Medical materials and uploaded information 542 may be entered at the disease, illness, or treatment stage with warnings and pop-up disclosures/disclaimers 544 as to not releasing confidential or private information to the patient UIX. If not diagnosed by a physician, the database proceeds to an inquiry 522 as to suggesting medical providers, seeking care/treatment, alternative suggestions (not medical advice).

If diagnosed by a physician, the next query allows a medical provider name 524 to be entered. The system queries internal libraries and external databases 525 to select the appropriate providers, associated medical systems, accepted insurance providers, licensure information, educational background, prior treated patients, prior diagnosis-disease associated symptoms, treatments, clinical trials, etc. After pulling in as much data as available and extrapolated, the system sends an email or communication 526 to the medical provider. Here, the medical provider may be a primary provider, surgeon, specialist, or otherwise, such that the communication is by way of email, patient portal, health system query, or other electronic messaging so that the physician/surgeon can directly access 528 the medical client-user profile that has been set-up and confirm, verify, or modify his/her information. Where some information may be selected to be non-modifiable data, a help button or live chat will be available to provide and answer physician questions. This more appropriately addresses professional needs, concerns, desires, suggestions, and provides greater accuracy in populating expertise in the medical profession. As well, the medical provider enters demographics, geographical data 540 including country, region, state, city, nearest metropolitan area, or as specified by country designations. Furthermore, a licensure validation 530 and confirmation will be configured to verify identities of medical providers. As such, local, state, national and international licensure requirements may vary and be modified/implemented in different ways and methodologies.

At various steps along the methodology, the system has full disclosure, electronic signature acceptance of terms and conditions in utilizing the social health network. Terms and conditions may vary based on patient client-user, physician client-user, and any person or entity/organization seeking a profile to establish or utilize the network communities. The educational background 532 of the physician/provider is queried manually to the user or to a database as to medical school attended/graduated, residency, fellowship, specialized education, licensure as to state, numbers, and as data is desired can be queried and coded, as appropriate. Experience data 534 is also included as to the physician/provider's employment status, medical or hospital affiliations, and any interconnected teams or centers 536 that are interconnected in real-time 538.

On a backend account, the physician client-user can verify an alias with an actual patient under his/her care and link to an electronic medical system/record (EMS/EMR). This disclosure is apparent to the client users even prior to setting up an account/profile. The medical provider/physician then can confidentially structure data in a database that will permit other medical providers access to information without complete identification of a patient. While this privacy/identification is often shared between different doctors and medical providers, this sharing of data and inadvertent use of such data by entities such as insurance companies, pharmaceuticals, and profit-bearing entities can be avoided by use of aliases in the system protected by the patient client-user. Therefore, physicians can interconnect with other physicians around the globe, access and track data of similar patients, or similar symptoms, behaviors, as selected and desired in a database. The interconnection of these physician based teams organizes ‘medical communities’ (similar communities of which are created on the patient accessible sites via the patient client-user accounts, as ‘patient defined communities’). While medical communities are accessible by physicians and medical providers (via secure access and encryption), the patient defined communities are open to anyone in the social health network (patient client users, physician client users, etc).

By way of example, profit-based organizations and corporations may set up profiles to better understand cost, analytics of care, treatment options, procedures aligned with costs, align entities with particular procedures/costs. Such entities may include insurance based industry 546, Medicare/Medicaid based government programs, pharmaceutical companies, among others. Such data and access to cost based data is owned by the social health network and accessible by way of controlled data uploads per requested configurations. Cost incurred 547, including co-pays, out-of-pocket, premiums, and otherwise will be included as desired. An upload or link to medical software 548 external to the network may push/pull data through an accessible interface, or API that integrates the software without release private, confidential, or personalized data (i.e., data to be de-identified and confirmed as de-identified prior to any import or export). Any personalized data or patient data will be de-identified (by way of alias or otherwise) and by permissive use and contractual arrangements to better understand global needs, personalized and public health awareness. The system will address privacy and security measures, and make client users aware of public disclosure of health information attributable and responsible to the patient-user, and that any information not intended to become a part of public medical record should not be disclosed; and that any data entered will be stored in databases, de-identified, and may be sold for use in public health diagnosis, treatment, studies, research, and across the medical and health care fields of use.

Any insurance-based use of data 549 is utilized internal to the system for cost analysis and AI to better serve healthcare operations, administration, costs, pharmaceutical purchases, biomedical device purchases, and any incentivized payment structures or commissions as to payouts to hospitals, providers, representatives, among others.

Communities established within the social health network are algorithmically determined and/or suggested by the system, or selectively personalized by a client-user, patient or medical provider. The client-user determines what his/her UI will share, or what is made viewable by other users. If a client-user wants to be included in future communities, has suggestions for such or otherwise, he/she can opt in or out. If client-user is a patient who simply desires to connect among family/friends, and create a health or support site, he/she may do that as well with minimal requirements to be entered into a shared database (e.g. disease/illness, symptoms, behaviors, activities, some basic medical info, and treatments, how long treated, planning, etc). This information has the benefit of epidemiological studies to allow trending data to be populated.

Continuing with the flow-chart of FIG. 5, a code 550 may be entered for an injury, illness, and/or disease to assist with algorithmic AI and deep learning for predictive analytics. Memory will assist in storing a set dictionary I (552) of data from external sources as to delegated and common names for disease, illness, treatment, medicines, and other material information.

Furthermore, FIG. 5 depicts a family-friend connection platform 554 that creates a social space to share with other users, patients or otherwise. The social connection or blog 555 will be tagged for use as metadata or searchable for key terms to create an information database, here dictionary II (556) for later analytics. The UI/UIX 557 created is what the user decides is viewed or shared, and by whom; the user decides what fields of data may be viewed publicly, privately, or via a select user community.

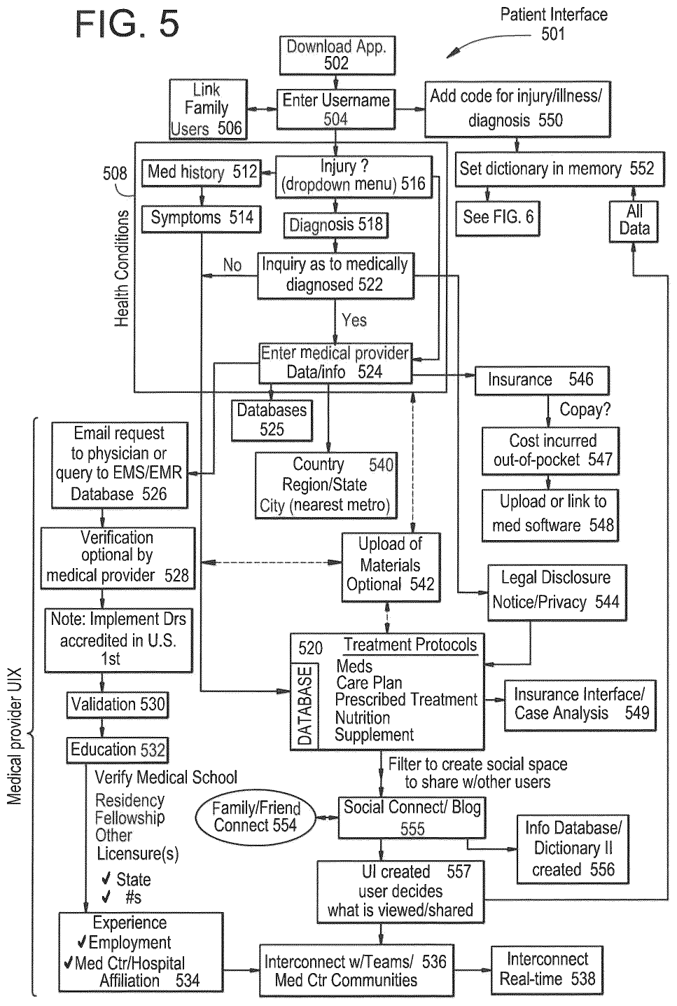

FIG. 6 implements a methodology as to entry and storage of patient data in the system 600. At the start 602 of the program, data/information from a patient client-user 604 (or client user who establishes a shadow patient user until the patient user verifies his/her account) is entered into the system and stored in memory 606. Other information from client-users specific to illness, disease, symptoms, etc, 608 as desired by prospective users may be entered. For exemplary purposes, and not limitation, a patient client-user with breast cancer seeking social interaction with other patients with breast cancer can select such affiliation. Specific disease, illness, treatments, etc thus are disclosed and stored 610 in order for the system processor to optimize profile alignments, as based on a matching mathematical model that suggests profile connections to a user 612. The client-user and prospect user access information about each other 614 (as desired and as available) to make a selection or de-select an online profile connection 616. Usage data 617 is stored as to social support network(s) established per disease, etc. Rating data is stored 618 as programmed and selected via algorithm refined 620 to rate and refine connections of client users and prospective users. Machine learning algorithms are utilized and models defined to use data of client users to provide a score and rating 622, and align or proposes connection to client users and prospective users. This information accumulates in the database 624. Predictive analytics of such data can be implemented to track patient information, data, and records around the globe, to assimilate Eastern and Western models of medicine for best treatment procedures and processes in personalized medicine (while protecting patient privacy and confidentiality, at the least, putting patients in control of their use of data, allowing an individual patient to determine what is and is not shared in the system in and shared networks). To the patient client-user, the benefit of socially interacting with others with similar disease and health conditions can be weighed against privacy and concerns for data.

Modifications in the data entry of FIG. 6 methodology allows the model defined 622 algorithmically to add or change information 626 about the client user, patient or doctor. This data may also be modified manually. Data as to the client user may be modified 628 as to additional diseases, ailments, illnesses, or changes status of disease, cure, or otherwise via the patient UIX, physician UIX, or algorithmically via data entries and patient portals of health systems such as EPIC or otherwise. An inquiry 630 asks a client-user, patient or physician whether that want to interact with prospective users 632. If no, the protocol ends 631. If the response is yes 632, the processing proceeds to matching mathematical model that suggests profile connections to a user 612. Modifications of the above may be customized and data entered, queries populated, as desired to conform to best practices in medicine and healthcare, and/or better treat and care for patients.

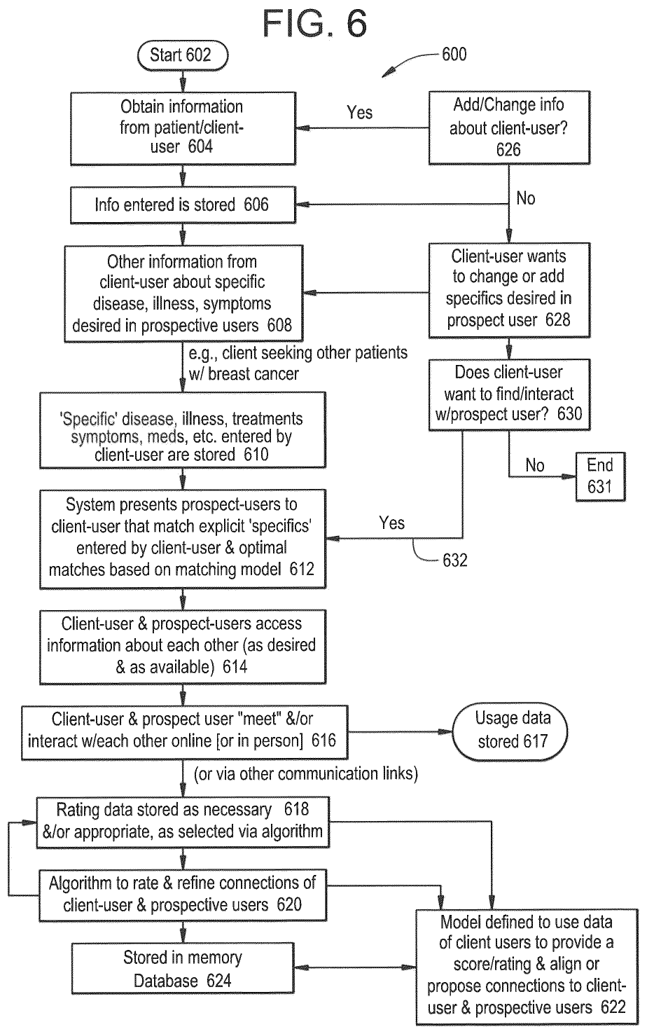

In FIG. 7, the flow chart demonstrates a medical doctor profile initiated 701. The social health networking application 700 includes a medical provider interface (UIX) 701. The medical provider may be implemented as any licensed medical professional, profiles established and associated with patients as patients designate 702 or as patients upload EMRs 703. The doctor may also refer or suggest 709a patient joining a medical community in the social network 700 via a patient UIX described prior. The medical doctor may establish a profile or be tagged/associated by the patient client-user in the system who answers the inquiry as to medical providers providing care. Here, an email 704 is sent to the doctor. If doctor declines, the system will continue to use the standardized information pooled from public databases 705 to populate the medical doctor profile account. As demonstrated here, if the doctor accepts the request, he/she will be directed to verify information 706, modify and confirm data 707 as to profile data 711, including, among others, licensure, society registries, publications, patents, journals, clinical trials associated with. The data is then synchronized 708 and updated in the physician UIX profile; and implemented at step 710 in the process. The doctor will further identify and confirm the patient 712 of the patient client-user with whom the email had been directed from. Where a patient does not opt into the system, a doctor may create a unanimous [coded/de-identified] patient profile 714 such that the later participating patient user would validate information in order to login and avoid duplication of an account or falsely create data. Information from the doctor verifies regional health systems and employers/hospitals that the doctor affiliates with. The medical doctor profile is created 716 is a medical provider licensed with the state or nationally/internationally. The doctor can access a public view 717 without patient EMR data and strictly what patient users have authorized. Otherwise, the doctor can access the professional portal 718 via the backend secure view where the doctor can login 719 and request connection 720 with other physicians in the global health social network 700.

Public view 717 of the physician profile may be similar to private, such as doctor background, licensure, etc. Patient data, if visible, from a physician or medical provider profile is de-identified by way of alias or otherwise. Physicians cannot pull direct patient identity from an EMR into the system, and if so, it is only viewable by other physicians. An encrypted login, possibly by way of digital certificate, allows a doctor to relate a specific EMR, or group of EMRs, to a customer number (e.g. number associated with a patient, or perhaps associated with a health system such as Lehigh Valley Medical System, or Oschner Medical System, or Willis Knighton Hospitals), and securely login to access identified patient data from the backend shadow patient profiles. The backend 718 shadow patient profiles are created by physicians who redirect EMR data and privacy information into a secure database 715 accessible only by licensed physicians, licensure as consented to by patients and providers of health systems to share in medical privacy information of patient in overall care and delivery of healthcare/medical services. As depicted, the secure database is a regional health system portal and/or employer/hospital database 715.

The queries 721 of the physician are directed to medical professionals secured in the network 700, diseases existing or unknown (and categorized symptomatically), treatments, medications, protocols, clinical trials, research and clinical studies, funded studies via government and industry, “zebras” (e.g. unknowns and rarities); symptoms 725, behaviors, episodes, history, among others are included variables and sources of data collection, without limitation. The diagnosis created may be generated via existing databases, and further queried and populated into databases via algorithmic data collection and analyses, machine learning and AI 726. Predictive outcomes 728, diagnoses, prognoses, as based on behaviors, doctor's profile in treatment, education, training, and geographical variables will be capable of being implemented in decision-making trees for improved patient care, outcomes, operational efficiencies and cost-effective measures.

As depicted in FIG. 7, communities 730 are created to align patients within a particular geography (i.e., city 731, state 732, country, continent, or other geography 733; or perhaps within a particular typical biome (e.g., rainforest, grassland, desert); or at particular global latitudes (e.g., equator-based diseases, death rates, depression; sub-tropical medical conditions; seasonal behaviors). Communities within the social health network may be patient designated communities, doctor or medical provider characterized communities, defined by med/pharmaceutical entity client-users, insurance client-users, government-based access, and any number of one or more of the listed, individually or in combination, varied and diversified. The communities may be suggested by learned algorithms of the system and recommendations for communities, or recognized trends communicated to client-users who would benefit from the community information or pooling of particularized data (data learned and client-users learned from deep learning based algorithms). Predictive analytics can help forecast future needs, anticipated openings in clinical trials, combinative treatment options, variations in acute and chronic treatment options, etc. Diagnoses may be addressed or even achieved where symptoms can be input and data sourcing optimized to relay prior treatments and success and/of failure rates. Episodic treatments and the movement of patients from one provider to another can also be tracked and care continuously provided for any illness and/or injury. Treatment communities 734 may be established manually or algorithmically through AI and deep learning to better understand treatment plans, personalized medicine, and alternative treatment options globally. In addition, medical device and pharmaceutical drug use 735 may populate communities for sharing parts and performance information (e.g. spinal implant groups, among others). Hospital system communities 736, disease-based communities 737, and patient-created emotional support communities 738, or otherwise may be created and established as determined by a user and led by patient or physician.

A community may request a patient user create a profile 739, request participation of a patient, or perhaps participation of another provider, physician or specialist.

In addition, surgical teams may find benefit here where teams operate on efficiencies in hospital operations and pay-for-service, e.g. anesthesia teams and anesthesiologists having access to patient care records, prior surgical records of a patient, prior operations, use of cannulas, medications, etc. Real-time video conferencing through the UIX with cybersecurity measures further allows a direct access to support networks, group chats, and face-to-face virtual meet-ups where immune systems are compromised and/or mobility limited to participate otherwise. As such, the medical provider profiles created would pull in patient data from patients of the surgeons, and invite/assemble medical teams, representatives of medical device and pharmaceutical companies, and operating room (OR) teams to better manage a patient and procedure effectively and efficiently under cost efficient and safety measures or concerns.

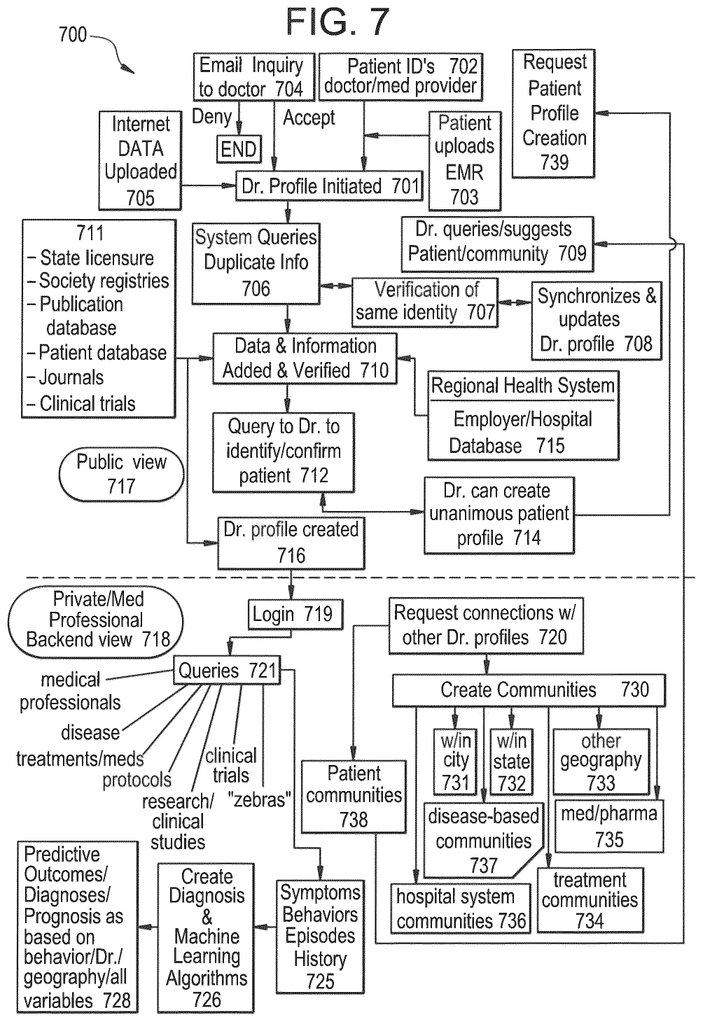

FIG. 8 demonstrates an embodiment of a system 800 that pools data from a physician and/or surgeon profile 801 including background education, medical schooling, residency, fellowship information, medical patients, outcomes, etc and streams that data into a shared database 805. Patient client-users 802 enter a profile with individual patient history, behaviors, etc. and view a disclosure authenticating use and permission to extract EMR 803 from patient record to the physician account/profile (identified/de-identified for sharing with other medical professionals and de-identified for profit-based healthcare or pharma/biomedical industry for purposes of enhancing healthcare delivery globally and providing of health services.

If permission is not granted by patient to extract EMR from hospital/provider network, the patient uploads or enters data and limited data is provided to verified medical provider confirmed databases and servers. A first database stores 807 this entry and use of data by a processor 811 to recommend connections. Data, images, medical info from the patient can still be uploaded 806 and provided for profile extrapolation.

Where patient client-user authorizes access of the system to his/her EMR for use in the health social system, the system provides notices 804, including a liability waiver, legal notices, privacy and compliance disclosures to be acknowledged 809 by patient client-user. Any data not entered via EMR may be entered 806 from patient uploads, and read via bots or algorithmic identifiers created to read EMRs. The patient client-users (whether including EMR or not) identify suggested and prospective users 810 by searching medical condition or disease or diagnosis (or any search request based on health care or condition). A processor recommends corrections and/or connection 811, communities 812, as based on data entries. Updates and verifications are affirmed 813. If at least one patient client-user or ‘shadow’ patient client-user account/profile, data is stored 815. Doctors and medical providers can recommend 816, refer, or create communications between patients 817, share postings or profiles, communities, etc. A separate professional/medical oriented database of medically licensed can establish backend secure communities 818 as well, and/or create personalized patient networks.

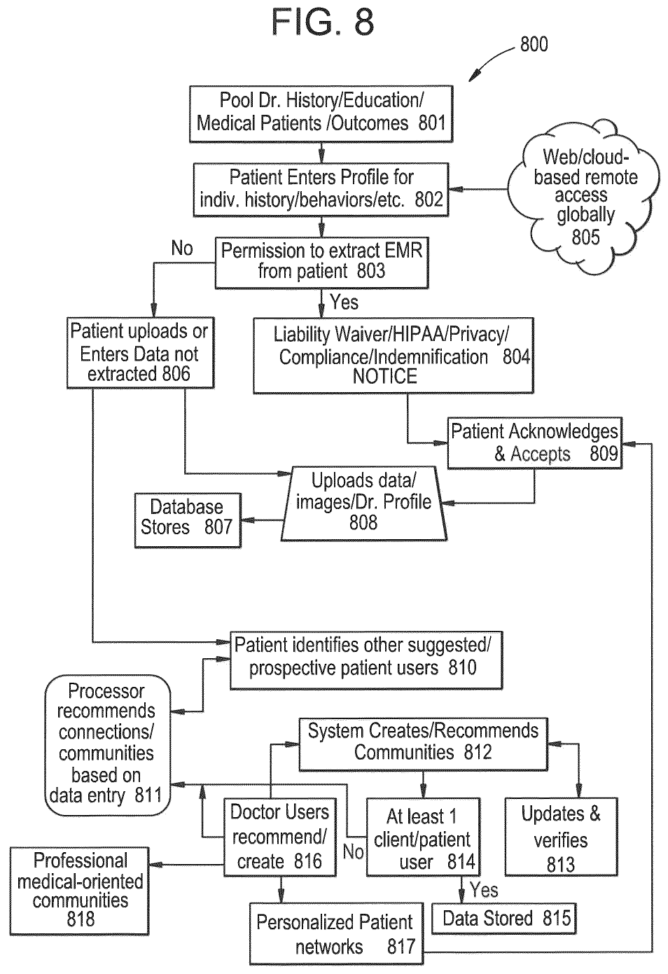

FIG. 9A is a value add-on to the system that allows communication to client-user profiles, here a doctor client-user 902, as to learned patterns and data structures how a client is using the system. Ads 903, 904, 905 and/or pop-up may be present on the UI of the profile, changing as based on client-user use. For exemplary purposes, and not limitation, the system may populate suggested medical facility data 906 related to one or more patient client-users. A notice may stream across the bottom of a screen as to clinical trial availability. Another ad may be suggested as to medical basis, disease, or learned processes and treatments; otherwise, a pop-up may provide outcomes globally suggesting particular treatment options or medicines. Pharmaceutical or medical device companies may advertise here, insurance display as to cost-basis and baseline medical treatment costs for procedures or particular medicines utilized by doctors.

FIG. 9B is particular to an embodiment of advertisements (ads) utilized at a patient client-user UI 910, including availability of facilities/hospitals offering active treatment programs 911, clinical trials 912, and/or pharmaceutical drug or device ads 913. Social events 914, ‘community’ fundraising opportunities within the network, on other online programs, or society based fundraising live events. Other ads and possibilities exist as to potential opportunities for value add, cost savings and efficiency in medical care services. Further, enforcement of regulations, e.g. Stark, Anti-kickback, FCPA, and preventions of fraud or misuse of data can be implemented and disclosed here and in profile based set-ups.

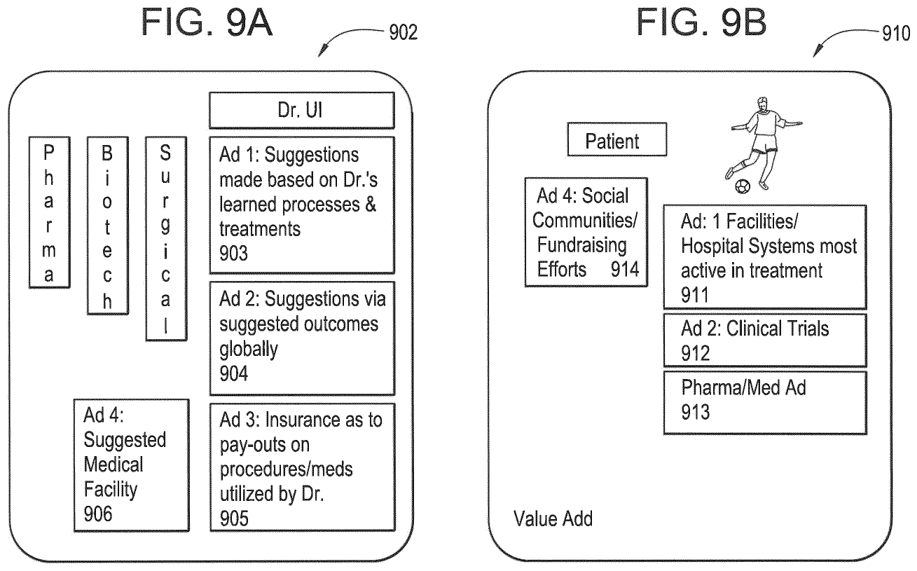

A family member client-user profile can associate and attach a patient profile as shown in FIG. 10. The family member may also be a friend, colleague, or otherwise. If the patient has not authorized or setup a patient client-user profile, then the family member client-user (or guardian type client-user) can establish a shadow patient profile to gather social and emotional support across a network, connecting with other family members who are in similar situations with children, aging parents, etc. The emotional support offered within the network among the caregivers of loved ones is also a substantive network of connections to bond, understand and better deal with watching a loved one/patient suffer, deal with pain, etc. Caregivers also seek emotional and support that medical and healthcare systems cannot adequately provide for unless displaying symptomatic and medically treatable conditions. The health social network here allows those caregivers to better connect and understand treatments, options, care, etc, emotional support and resources that often get overlooked in the primary care of the injured/diseased patient.

Note that FIG. 10 also allows the system 1400 to query and to sort most relevant data as to trends, reoccurrences, increasing geographies or spatial relations globally, recommending vaccinations, treatments, etc. Perhaps the family client-user 1402, here, is a parent of a child who has now reached his/her 18^{th} birthday 1405 in the United States (other considerations of ‘adult’ provided for as based on country of other users). The owner of the shadow patient account 1402 receives a notice and request to transfer or discard patient related information. If discarded, the data remains de-identified in a database 1406. If transferred, the system generates communication or recognizes a profile entry of a new adult patient client-user 1403 as synchronized with prior account set-up 1407. Note that birthdate would need be a confirmed entry by a medical provider at time of profile set-up by patient client-user who pulls in EMR data, the birthdate later being unmodifiable. After a notification period, e.g. 90 days prior to shadow patient user account aging 18 years, the account would invalidate parental or family member access to that account. Otherwise, a shadow account would become inactive unless reconfigured via a link in the system and verified identities. Again, separate databases would ensure verification and validation of various forms of data.

In system 800 of FIG. 10, a query 1404 into symptoms of the patient generates a machine-learned algorithmic result or solution 1408 to sort relevant data 1410 as to closest proximity of ailments geographically, ranking seasonal concerns, recommending timeframes for vaccinations/treatments, and uploading physician UIX 1411 info into the updated database. In combination with converting an adult profile 807, the new profile is capable of synchronizing old records 1412.

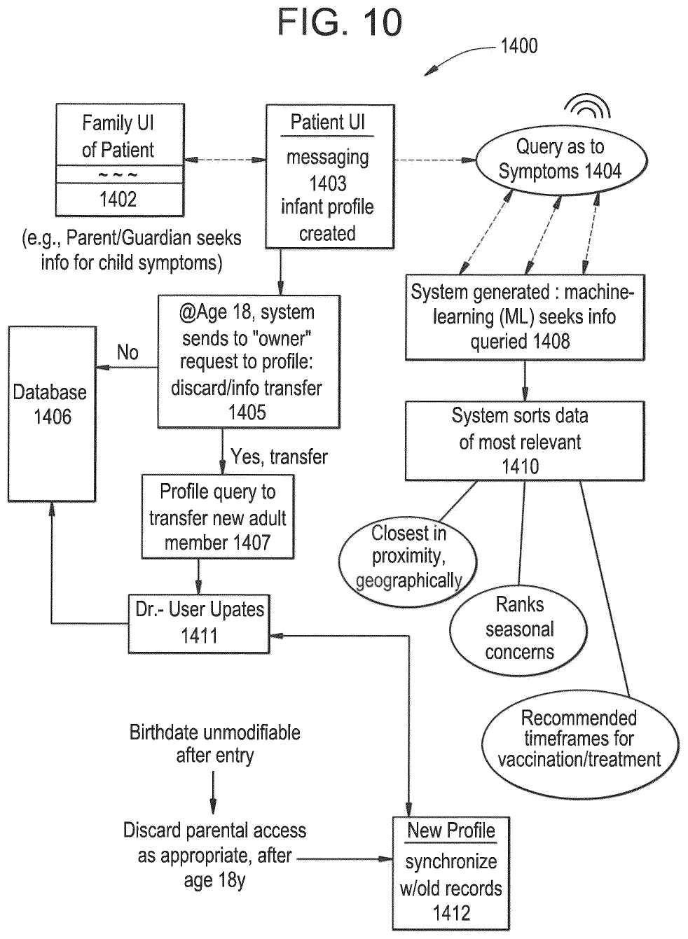

FIG. 11 depicts an embodiment of a doctor profile 902 with image, provider demographics 933, including name, degree, profession, address and other contact information as appropriate. Hospital affiliations, education, training, awards, and resume/CV (curriculum vitae) upload are apparent. Years in practice, areas of interest, trials, publications, insurance programs also are listed. Resume upload is sortable, compliable and capable of being scanned for data.

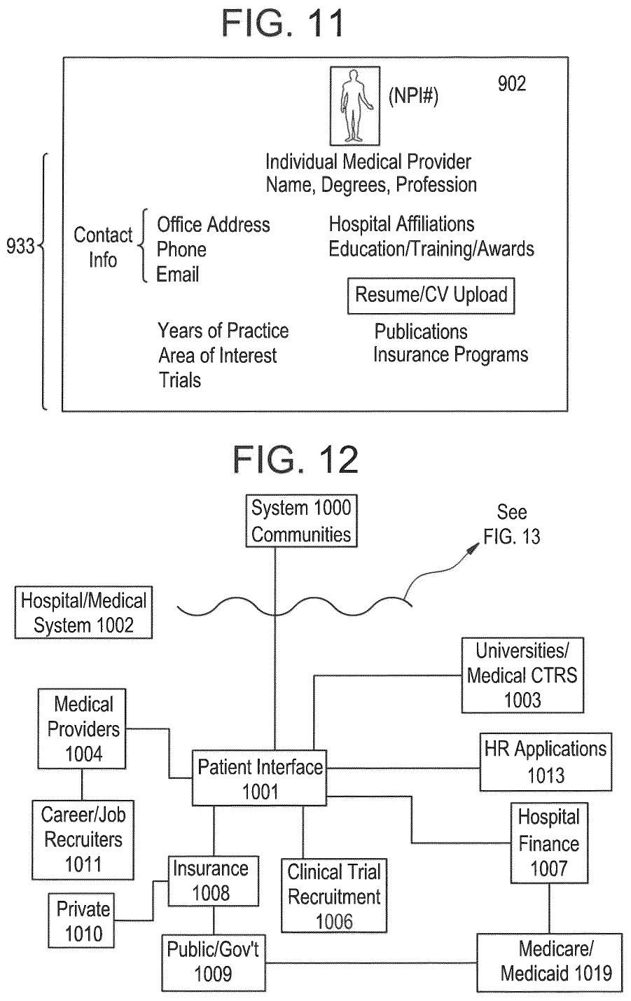

FIG. 12 demonstrates various examples of system communities 1000 without limitation. Such communities are built around a patient client-user interface 1001 to include hospital/medical system communities 1002, university medical centers 1003, medical provider communities 1004 (linking to career/job recruitment), insurance communities 1005, (private and public), clinical trial recruitment 1006, hospital finance 1007, insurance communities 1008 including Medicare/Medicaid (government) access communities 1009, and private insurance 1010, human resource communities 1011 for much needed clinicians, doctors, nurses, among others.

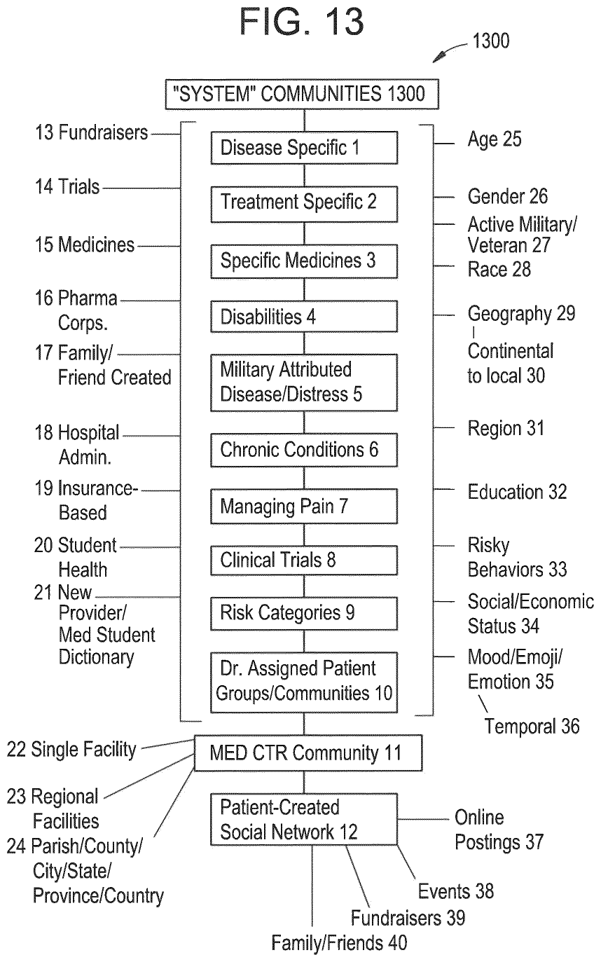

FIG. 13 further sets out the system communities 1300 as defined in the online and cloud-based social health network. The communities may be specific to disease 1, treatments 2, medicines 3, disabilities 4, military/war activities 5, travel, chronic conditions 6, management of pain 7, clinical trials 8, risk categories 9, behavioral risks, doctor-assigned patient groups 10, patient created social networks 12, caregiver support groups. The provider based system demographics range from medical schooling, prior treatments/care provided, facilities, geographic location, seasonal demographic, biome, pharma, trials, fundraisers 13, new treatment options, Eastern and Western medical practices. The patient demographics also looking at age 25, gender 26, military/veteran service 27, race 28, geography 29, continental/local 30, region 31, education 32, risk/behaviors 33, activities, socio-economic status, mood 35, emoji/emoticons included with emotions (i.e. temporal and trending via treatment course 36). The UIs of medical provider and/or patient client-users will be similar to functionality of commonly used social networks today, including and not limited to, image uploads, status changes, events, activities, relationship associations (here linked not only by family or personal relationship status, but also linked to patient relationship, possibly ‘caregiver’ status). In addition, the system may include Xray or MRI uploaded information or slices of CT imaging, blood sample results, testing, etc. A patient may upload to his/her preferences. The EMR portion, however, restricted to medical provider access. Any restrictions and/or limitations may be modified in context of regulatory measures and laws across the globe.

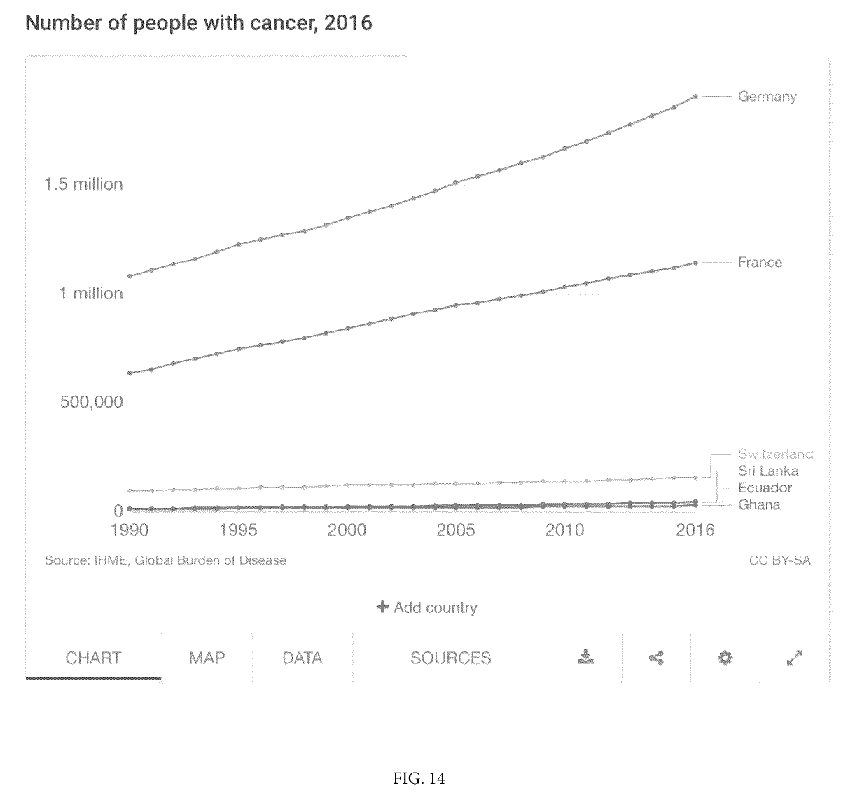

FIG. 14 is a graphical depiction of disease regionally, and spatially in time, targeted to a specific disease. The graph here indicates the number of people with cancer within 2016 and across regions, e.g. Sri Lanka and others (as demonstrated by the different linear transgressions in the graph.

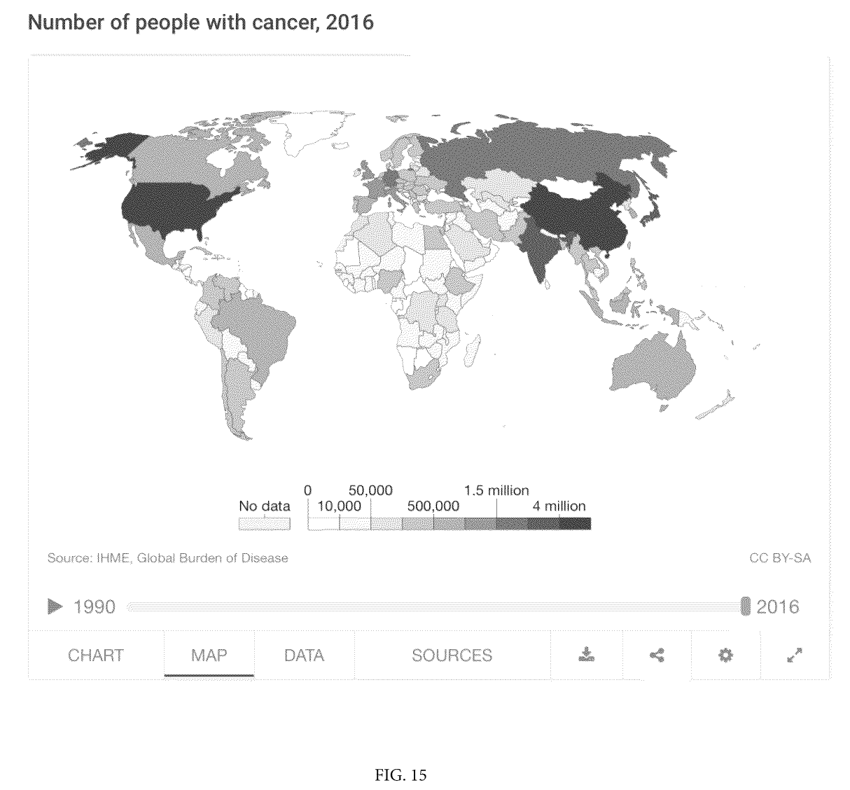

FIG. 15 represents an embodiment in a geographical depiction representing aspects of the information defined herein. Specifically, the depiction is a map that generates data in an illustration for easy translation of the data to a user. The user can then deduce information to support decision-making for seeking care, or finding the most efficacious treatments (such charting and depictions also capable of being depicted in such format.

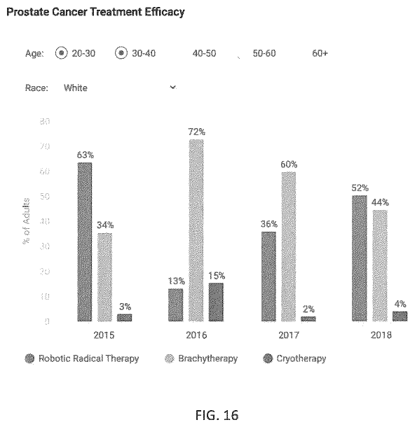

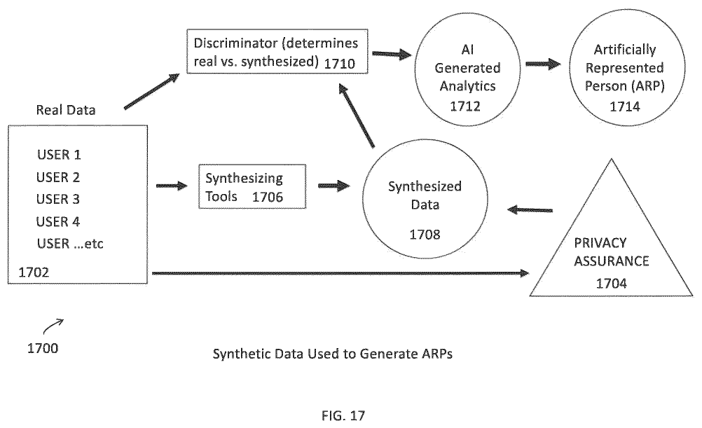

FIG. 16 represents an embodiment in a depiction of efficacy of treatment aligned with demographics in one aspect. Here the success rates of robotic radical therapy, brachytherapy, and cryotherapy are contrasted across the selected timeframe of 2015 through 2018 for all persons ages 20-30 of the white ethnicity. The treatment efficacy is generated from real person data, specifically from patient records verified across electronic medical records (EMRs) of health provider systems. Any inquiry may be searched and terms relevant of interest to a user to integrate predictive learnings, machine learning, and/or artificial driven analytics to generate a response in typographical form such as text or in a designated depiction of graph, chart, mapping, decision tree, or otherwise. Generative AI with large language models (LLMs) are integrated, such as, for example and not limitation Open AI's ChatGPT. Such LLMs may be customized within a domain of data or to preserve proprietary data in a closed network. Such data may utilize a Discriminator 1710 or other data verification tool, (similar to that shown in FIG. 17) to validate the data and ensure accuracy and specificity for vertical industry use (within the field of art). Generative artificial intelligence (AI) may automate tasks, create ideas, generate suggestions as programmed, and code software from learned patterns and recognition of data. As such, the data will be better utilized clean and free of inaccuracy, specific to the targeted request. Bots may also be artificially generated to track user data and create synthetic sets of data that duplicate profiles of a user, of a user's affiliation or digital communities. In creating such synthetic datasets, the digital platform may implement the discriminator to delineate real person vs. synthesized data.

As demonstrated in FIG. 17, a digital platform 1700 incorporates a plurality of users and real data user profiles 1702. The real data 1702 is protected through privacy assurance 1704 to protect PII, and also PHI as later integrated. Synthesizing tools 1706 and algorithms create synthesized data 1708. The synthesized data 1708 can be dual authenticated through privacy assurance 1704, as desired. Real data 1702 and synthesized data 1708 get pushed to a discriminator 1710 to determine and distinguish real vs synthesized data at a current or future data, and stored in an isolated or shared database. A processor is programmed to generate artificial intelligence (AI) generated analytics 1712, the synthesized data and AI analytics creating an artificially represented person (ARP).

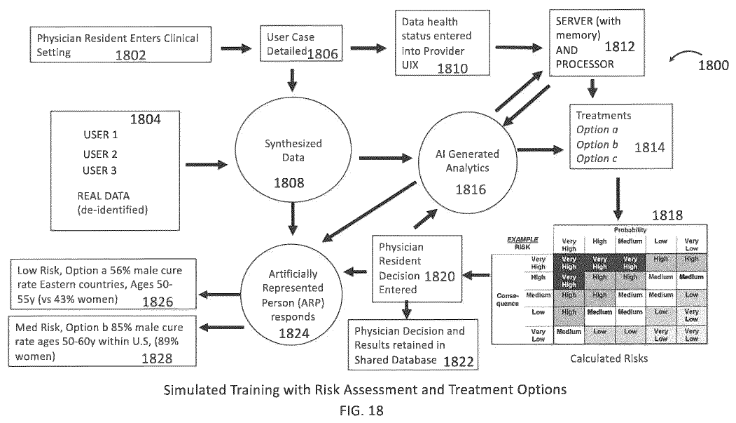

FIG. 18 illustrates an embodiment that creates real and simulated treatment models, alone or in combination. The simulated training model 1800 is initiated by a health provider 1802, such as a physician resident within a medical school setting. The health provider 1802 takes the details of a patient, here a user of the digital platform and the user case 1806 is recorded. The health status data of the user is entered into the digital platform 1800 through a provider user interface (UIX) 1810. The provider UIX integrates with the shared database generated from the user profiles in platform 1700, but the provider UIX is separately served by a secondary server. The provider system and UIX accesses the digital platform but is a discrete access tool that may or may not allow a provider to engage with the user or user communities. Once health status data is entered, the secondary server 1812 with processor utilizes AI generated analytics and code to create treatment options (a, b, c, . . . etc.) 1814 from the health status data entered. The health status may be mental/psychological state or physical condition, disease, therapy, treatment plan, surgery status, among any other state associated with health and wellness of an individual. The health status may integrate personal data from digital and social platforms, from digital engagements online or in use of electronic/mobile devices. Health status may be pulled in from wearables (e.g. iPhone Apple watch, or monitoring device) or an implant, or other medical device system (e.g. imaging such as MRI, CT, Xray, PET, etc), directly or indirectly through third party based software. Treatments 1814 and risk assessments (e.g., low, medium, high, or other rating system) of the probability and consequences are generated in a table of calculated risks 1818. Once the health provider enters his/her decision 1820, the results are stored in the shared database 1822 and also utilized to create an artificially represented person (ARP) that is aligned with success rates and efficacy of treatments 1826, 1828. Here, for example the ARP is generated from either synthesized data 1808 or through real person users at the user case detailed step 1806, and the efficacy and alignment of risk ascertained. In one aspect, the ARP has a low risk decision 1826 if the user is a male between the ages of 50-55 years in an Eastern country such as the Philippines with a cure rate of about 56% (or 43% cure rate if a woman of similar age). In another aspect, medium risk 1828 is accessed for choosing Option b treatment planning with an 85% success rate if a male between the ages of 50-60 years or 89% cure rate if a woman. These risk and efficacies of treatment planning may be further narrowed as search, and queried in the digital platform. The presentation of data and risk/efficacy decision chart may also be generated in mapping, graphical, charting or other creatively designed depiction at the user interface or provider interface.

As demonstrated the simulated training 1800 can be utilized for the general user (patient) to simulate decision-making and determining health assessment and risks associated with treatment plans determined from real and/or synthetic data. The medical or health provider setting, however, is better aligned for creating a simulated training with risk assessment, treatment options, safety to patient, and integrating other environmental factors to create a personalized training model. The training models can be created to reduce training costs and reduce expenditures on resources as well. The models also create a safe mechanism of training for future care and procedures if the planning, surgery, or therapy can be modeled by billions or trillions of data points, both real and synthesized. Furthermore, federal and international databases can be integrated as resources and tools in the network for the social network user. Such databases may include for example and not limitation, the NCCIH Clearinghouse (providing information on NCCIH and complementary and integrative health approaches; www.nccib.bih.gov), PubMed® (a service of the National Library of Medicine comprising publication information from scientific and medical journals; www.ncbi.nlm.nih.gov/pubmed/.node), the Cochrane Database of Systematic Reviews (evidence-based reviews produced by the Cochrane Library, an international nonprofit organization summarizing results of clinical trials on health care interventions; www.cochranelibrary.com), NIH Clinical Research Trials and You (website created by NIH to help people learn about clinical trials, why they matter, and how to participate; www.nih.gov/health/clinicaltrials/.node), Research Portfolio Online Reporting Tools Expenditures & Results (RePORTER) (a database of information on federally funded scientific and medical research projects being conducted at research institutions; www.projectreporter.nih.gov/reporter.cfm).

The electronic system, a social health network disclosed herein, interconnects patients, families and friends of loved ones, who desire information upon diagnosis to properly resource their needs for knowledge of not only the disease, but including personalized connections to others who suffer and heal similarly, to caregivers who also demand emotional support. The individuals of the social network of patients will beneficially be able to share their experiences publicly across the network to interconnect with others, interconnect with medical professionals globally, and/or obtain comparisons in medicine from other patients and/or medical professionals. As well, the medical professional network allow a doctor, surgeon, nurse or licensed medical provider to expand their knowledge base as to diagnosis and treatments across global medical communities. The social health network beneficially details the possibilities of solving the needs for social support networks in medicine, the interaction of medical professionals and access to global patient data, while implementing the technological measures to achieve the same. The system goes beyond expectations for care and treatment of patients, providing a support network for caregivers, and also revolutionizing the training and expertise of medical professionals. As well, escalating costs for healthcare services can be addressed in a more analytically driven methodology, providing consistency and efficiency in medicine. With a roaming population of individuals, patients, caregivers, and medical providers, travel has demanded access to patient care that is not currently provided; the social health network described here provides that capability. Medical providers and patients can similarly access and seek medical treatment and care globally, including support networks and emotional ties and connections that no other system has readily made available in healthcare.

In addition, methods disclosed herein analyze social method of analyzing social interactions to present content of interest to a user as selected by a recommendation unit. Much like the various social apps, this program is designed to encourage social interactions among users with similar interests and health conditions. When using a social networking system and viewing a webpage that includes information provided by the system, social interactions are allowed and recommend interconnecting. Certain types of social interactions are monitored and detected, recommending particular connections and identifying users based on a description of the interaction desired. The recommendation suggests the user engage another user in the social health networking system per health-based classifications. Any modification of information or use of the above may include any number of variables to be implemented and modified to achieve the same and does not depart from the spirit and scope of the disclosed invention.

What is claimed is:

1. A digital platform comprising:
one or more servers including a plurality of health data;
one or more processors programmed to create profiles of one or more users, each profile including data tailored to a designated person, wherein the data includes parameters input by the user and extracted data from digital profiling, wherein the parameters comprise user demographics and health status; and
a shared database directed by the server to store the data and the updated data;
wherein the server is programmed to: (a) receive a request from the user at a user interface to create or join at least one online patient community categorized by one or more health conditions, (b) receive a first request from the user to delineate personal identifiable information (PII) and de-identify the PII as de-identified data, (c) receive a second request from the user to authorize release of the de-identified data to the shared database, and (d) access the data by way of the processor, wherein the processor uses machine learnings and artificial intelligence (AI) driven analytics to predict outcomes, treatments, and disease progression using user-derived data.

2. The digital platform of claim 1, wherein the health status comprises symptoms, complaint, injury, health condition, health status, disease-state, treatment, surgery, therapy, or medication of the user, or a combination thereof.

3. The digital platform of claim 1, wherein the extracted data comprise social interactions, digital engagement across cloud-based platforms, metadata, cookies, reactions to digital media content, time spent with online communities, data entered by the user from the electronic medical record (EMR), and data from medical devices, wearable devices or implanted devices, the processor further programmed to update the data.

4. The digital platform of claim 1, wherein communication between users occurs at the user interface external to a health provider portal.

5. The digital platform of claim 1, wherein the user allows selecting or deselecting the extracted data configured to be integrated in the analytics of the processor.

6. The digital platform of claim 1, wherein the plurality of health data comprises data input by a plurality of users and data aggregated from external resources, clinical studies, and clinical practice.

7. The digital platform of claim 1, wherein the one or more processors is further programmed to create an AI profile, the AI profile comprising synthetic data artificially generated from real patient data to create the AI profile.

8. The digital platform of claim 7, wherein the AI profile represents an artificially represented person (ARP).

9. The digital platform of claim 1, wherein the parameters input by the user comprise an election of one or more licensed health providers associated with the user.

10. The digital platform of claim 9, wherein the licensed health provider comprises a care provider including an individual or a system entity comprising: medical doctors, doctors of osteopathic medicine, dentists, specialists, surgeons, nurses, physician assistants, dental assistants, alternative care providers, aestheticians, hospital systems, health-affiliated educational institutes and organizations, managed care living facilities, mental health organizations, and other care provider groups.

11. The digital platform of claim 9, wherein the user digitally submits a request for an electronic medical record (EMR) from the licensed health provider, the user authenticates the request, and the licensed health provider directs an uploaded version of the EMR to the user interface of the digital platform.

12. The digital platform of claim 9, wherein the licensed health provider is provided a digital request to engage with the digital platform at a secondary user interface, and a secondary processor accesses the shared database to drive predictive analytics at a secondary server exclusive to a plurality of licensed health providers.

13. The digital platform of claim 12, wherein the licensed health provider inputs at the secondary user interface one or more of provider demographics, education, residency, or clinical experience, and authorizes release of de-identified medical data from persons or machines to the shared database.

14. A method of using the digital platform of claim 1, comprising:
initiating the user profile;
defining a relationship between a plurality of users and the health status;
creating one or more communities based on the demographics of the users, the user inputs, the health status, and associated with one or more health providers; and
selecting search terms to generate artificial intelligence (AI) driven response to guide decision-making.

15. The method of claim 14, wherein the data from the user profiles is de-identified to synthesize simulated health conditions.

16. The method of claim 15, wherein the processor generates a plurality of synthetic data from profiles.

17. The method of claim 16, wherein the synthetic data creates at least one AI profile that represents an artificially represented person (ARP).

18. The method of claim 15, wherein the simulated health conditions create hypothetical clinical experiences to engage with a trainee.

19. The method of claim 18, wherein the trainee elects one or more treatments to structure a care plan for the user with a specified health condition.

20. The method of claim 15, wherein the simulated health conditions are structured in an artificial intelligence (AI) profile, and the AI profile responds to one or more AI generated treatment plans, and wherein the one or more processors generates, from the AI profile, graphical depictions representing risk and success rates corresponding to the AI generated treatment plans.
